(19) United States
(12) Patent Application Publication
(10) Pub. No.: US 2024/0252773 A1
(43) Pub. Date: Aug. 1, 2024

(54) PHLEGM SUCTION ALARM ISSUING APPARATUS AND METHOD THROUGH MONITORING R ESPIRATORY SOUND OF TRACHEOSTOMY PATIENT IN REAL TIME

(71) Applicants: THE CATHOLIC UNIVERSITY OF KOREA INDUSTRY- ACADEMIC COOPERATION FOUNDATION, Seoul (KR); INDUSTRY-ACADEMIC COOPERATION FOUNDATION, YONSEI UNIVERSITY, Seoul (KR)

(72) Inventors: Young Hoon JOO, Seoul (KR); Hyun Bum KIM, Seoul (KR); Jong Baeg KIM, Seoul (KR); Yo Han JUNG, Seoul (KR); Dae Yeon KOH, Seoul (KR); Min Hyeong KIM, Seoul (KR); Hyun Jun HAN, Seoul (KR)

(21) Appl. No.: 18/013,323

(22) PCT Filed: Aug. 11, 2022
(86) PCT No.: PCT/KR2022/012017

§ 371 Date: Dec. 28, 2022

(30) Foreign Application Priority Data
May 31, 2022 (KR) 10-2022-0066684

(51) Int. Cl. A61M 16/04; A61B 5/00; A61B 5/08

(52) U.S. Cl. CPC A61M 16/0472; A61B 5/0816; A61B 5/7203; A61B 5/7257; A61B 5/743; A61M 16/0463; A61M 2205/18

(57) ABSTRACT

Proposed are a phlegm suction alarm issuing apparatus and a method for a tracheostomy patient which monitor and analyze a respiratory sound of the tracheostomy patient in real time, then check congestion of phlegm, and issue a phlegm suction alarm.

### TECHNICAL FIELD

The present invention relates to both phlegm suction alarm issuing apparatus and method for a tracheostomy patient, and more specifically, to both phlegm suction alarm issuing apparatus and method for a tracheostomy patient which monitor and analyze a respiratory sound of the tracheostomy patient in real time, then check congestion of phlegm, and issue a phlegm suction alarm.

### BACKGROUND ART

Increases in aging population and chronic disease result in an increase in respiratory disease such as stroke or aspiration pneumonia, and the performance of tracheostomy or tracheotomy for such diseases is also increasing accordingly.

Since the tracheostomy is performed at a low part of an upper airway, the upper airway is not able to completely fulfill both humidification and dust removal functions, and thus more phlegm is produced than usual.

Accumulation of phlegm in a tracheostomy tube results in changes in shape and mechanical characteristic of a passage (tracheostomy tube and trachea of a patient) through which air flows, thus resulting in a change in respiratory sound. In addition, a patient who has a feeling of a foreign body in the air passage even intentionally adjusts a breathing pattern.

When phlegm having strong fluid-like properties is much accumulated, a trembling sound (phlegmy sound) having a low frequency is produced. When phlegm having strong solid-like properties is much accumulated, a cross-sectional area of the air passage decreases, and a sound (wheeze) having a high frequency is produced.

Since a medical team cannot check an amount of phlegm with the naked eye, the medical team discriminates between a normal respiratory sound and a respiratory sound produced in a highly phlegmy condition by checking a respiratory sound of a patient and determines a phlegm suction time and an internal tube replacement time when phlegm is accumulated in a tracheostomy tube and cannot be removed through suction.

When phlegm is not properly removed, the tracheostomy tube is congested, and congestion of phlegm results in respiratory failure or secondary pneumonia.

Hence, phlegm suction is performed periodically at intervals, or suction is performed when a phlegmy sound is determined to be loud with frequent observation. Further, it is not possible to check a phlegm removing time for a patient who is unconscious or has difficulty in communicating, and thus a medical team has to perform regular suction every one to two hours or has to perform suction by hearing a phlegmy sound.

Even in this case, randomly frequent suctions without checking an amount of phlegm can include unnecessary suction such as suction depending on time intervals even when phlegm is not accumulated. The unnecessary suction results in problems of giving uneasiness to a patient and simultaneously damaging a tracheal wall or causing a painful cough.

In addition, a problem arises in that a nurse, a caregiver, or a guardian has to watch 24 hours to properly remove phlegm.

### PATENT LITERATURE

Korean Patent Registration No. 10-2314270

### SUMMARY OF INVENTION

### Technical Problem

The present invention is made to solve the above-described problems of the related art, and technical objects thereof are to provide both phlegm suction alarm issuing apparatus and method for a tracheostomy patient which monitor and analyze a respiratory sound of the tracheostomy patient in real time, then check congestion of phlegm, and issue a phlegm suction alarm.

Technical objects to be achieved by the present invention are not limited to the technical objects mentioned above, and the following description enables other unmentioned technical objects to be clearly understood by a person of ordinary skill in the art to which the present invention pertains.

### Solution to Problem

In order to achieve the above-described objects, an embodiment of the present invention provides a phlegm suction alarm issuing apparatus including: a body that is coupled to an entrance of a tracheostomy tube and an external tube and forms a respiratory channel for a patient; an interior microphone that is attached to an inner wall of the respiratory channel of the body and extracts a respiratory sound of the patient; a flow rate sensor that is attached to the inner wall of the respiratory channel of the body and measures a respiratory flow rate of the patient; and a suction alarm control unit that extracts a frequency of the respiratory sound of the interior microphone based on information of the respiratory flow rate, analyzes frequency characteristics of the respiratory sound, and then outputs a phlegm suction or internal tube replacement alarm signal when the frequency and an abnormal respiratory cycle or flow rate data which are stored in advance are compared and are equal to each other within a certain frequency or amplitude range.

The phlegm suction alarm issuing apparatus may be configured to further include a waterproof member that seals the interior microphone in order to waterproof the interior microphone.

The phlegm suction alarm issuing apparatus may further include an exterior microphone that is attached to an exterior part of the body and extracts an exterior voice sound, and the suction alarm control unit may be configured to remove noise contained in the respiratory sound of the patient by removing the exterior voice sound extracted by the exterior microphone from the respiratory sound extracted by the interior microphone.

The body may be configured to include a tracheostomy tube coupling flange that has a cylindrical flange which is coupled to an entrance of the tracheostomy tube and one or more support flanges which project from an outer surface of the cylindrical flange and support the body on the tracheostomy tube; and an external tube inserting pipe which extends from the cylindrical flange and into which an external tube is inserted and coupled.

The body may be configured to further include an LED unit that emits light for a suction or internal tube replacement alarm; and a speaker unit that outputs a sound for a suction or internal tube replacement alarm.

The body may be configured to further include a power button for power on and off and a charging terminal for charging an internal battery.

In order to achieve the above-described objects, another embodiment of the present invention provides a phlegm suction alarm issuing method that is performed by a phlegm suction alarm issuing apparatus including a body having a respiratory channel for a patient, an interior microphone and a flow rate sensor attached to the respiratory channel, and a suction alarm control unit which analyzes a respiratory sound extracted by the interior microphone and a respiratory flow rate extracted by the flow rate sensor and outputs a phlegm suction or internal tube replacement alarm, the phlegm suction alarm issuing method including: a collecting step of detecting and collecting the respiratory sound and the respiratory flow rate of the patient through the respiratory channel by the suction alarm control unit using the interior microphone and the flow rate sensor; a respiration time extracting step of extracting a respiration time from the respiratory sound based on information of the respiratory flow rate by the suction alarm control unit; a frequency characteristic analyzing step of analyzing a frequency characteristic of respiration; an abnormal respiration or flow rate detecting step of detecting an abnormal respiration cycle or flow rate when analysis information of the frequency characteristic of the respiration analyzed in the frequency characteristic analyzing step and analysis information of a frequency characteristic corresponding to phlegm suction or internal tube replacement stored in advance are compared and are equal to each other within a certain frequency or amplitude range; and an alarm outputting step of outputting an alarm of the suction time or internal tube replacement time when an abnormal respiration or flow rate is detected in the abnormal respiration or flow rate detecting step.

Here, the phlegm suction alarm issuing apparatus may further include an exterior microphone that is attached to an exterior part of the body and extracts an exterior voice sound, and the phlegm suction alarm issuing method may further include, after the collecting step, a noise removing step of removing noise contained in the respiratory sound of the patient by the suction alarm control unit by removing the exterior voice sound extracted by the exterior microphone from the respiratory sound extracted by the interior microphone.

### Advantageous Effects of Invention

According to the above-described embodiment of the present invention, a medical team or a guardian is notified of a phlegm suction time or an internal tube replacement time by monitoring a respiratory condition of a tracheostomy patient, and thereby the embodiment is effective in that a remarkably easy phlegm suction is performed.

In addition, according to the embodiment of the present invention, a medical team or a guardian is notified of a phlegm suction time or an internal tube replacement time by monitoring a respiratory condition of a tracheostomy patient, and thereby the embodiment is effective in that the medical team or the guardian is relieved from continuous observation on the patient at a close position for the phlegm suction.

In addition, according to the embodiment of the present invention, a medical team or a guardian is notified of a phlegm suction time or an internal tube replacement time by monitoring a respiratory condition of a tracheostomy patient, and thereby the embodiment is effective in that of a problem of a damage to a tracheal wall or a painful cough caused by randomly frequent suctions without checking an amount of phlegm in the related art is solved.

Effects of the present invention need to be construed not to be limited to the above-mentioned effects but to include every effect that can be derived from configurations of the invention described in the detailed description and claims of the present invention.

### BRIEF DESCRIPTION OF DRAWINGS

FIG. 1 is a perspective view of a phlegm suction alarm issuing apparatus 1 for a tracheostomy patient according to an embodiment of the present invention.

FIG. 2 is a view illustrating a waterproof member of an interior microphone 170 in FIG. 1.

FIG. 3 is a functional block diagram of a suction alarm control unit 300 in FIG. 1.

FIG. 4 is a view illustrating a procedure of coupling the phlegm suction alarm issuing apparatus 1 for a tracheostomy patient to a tracheostomy tube 10 according to the embodiment of the present invention.

FIG. 5 is a flowchart illustrating a processing procedure of a phlegm suction alarm issuing method according to the embodiment of the present invention.

FIG. 6 is a graph illustrating (a) sound signal, (b) spectrogram, and (c) flow rate signal based on time which are analyzed in a frequency characteristic analyzing step S40 in FIG. 5.

FIG. 7 is a graph illustrating (a) signal magnitude based on time, (b) spectrogram visually displaying a change in frequency based on time through the short-term Fourier transform (STFT), and (c) signal magnitude based on a frequency converted through the fast Fourier transform (FFT) according to each type of respiratory sound.

FIG. 8 is a view illustrating a noise removing step S20 performed by a noise removing unit 310 in FIG. 5.

### DESCRIPTION OF EMBODIMENTS

In the following description of the present invention, when the detailed description of a known function or configuration related to the present invention is deemed to result in obscuring the gist of the present invention unnecessarily, the detailed description thereof will be omitted.

Embodiments according to the concept of the present invention can be variously modified and can include various examples, and thus specific embodiments are to be illustrated on the drawings and are to be described in detail in this specification or this application. However, this does not mean that the embodiments according to the concept of the present invention are limited to a specific disclosed example, but is to be construed to mean that the present invention includes every modification, equivalent, and alternative included in the technical idea and technical scope of the present invention.

The description in which one configurational element is “connected to” or “coupled to” another configurational element is to be understood to mean that the one configurational element can be directly connected to or coupled to the other configurational element, or still another configurational element can be present therebetween. On the other hand, the description in which one configurational element is “directly connected to” or “directly coupled to” another configurational element is to be understood to mean that no configurational element is present therebetween. The same is true for other terms, that is, “between” and “directly between”. “adjacent” and “directly adjacent”, or the like for describing relationships between configurational elements.

Terms used in this specification are only used to describe a specific embodiment and are not intentionally used to limit the present invention. A word having a singular form also includes a meaning of its plural form unless obviously implied otherwise in context. In this specification, terms such as “to comprise” or “to include” are to be construed to specify that a feature, a number, a step, an operation, a configurational element, a member, or a combination thereof described here is present and not to exclude presence or a possibility of addition of one or more additional features, numbers, steps, operations, configurational elements, members, or combinations thereof in advance.

Hereinafter, the present invention will be described in more detail with reference to the accompanying drawings illustrating embodiments of the present invention.

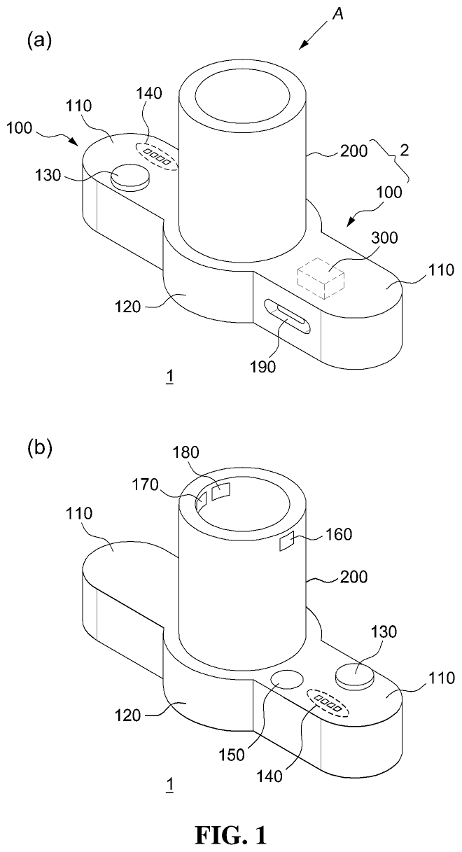

FIG. 1 is a perspective view of a phlegm suction alarm issuing apparatus 1 for a tracheostomy patient according to an embodiment of the present invention.

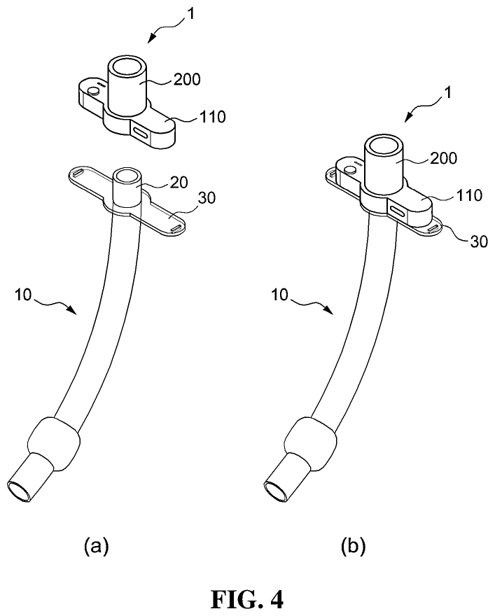

As illustrated in FIG. 1, the phlegm suction alarm issuing apparatus 1 can be configured to include a body 2 that is coupled to an entrance 20 (refer to FIG. 4) of a tracheostomy tube 10 (refer to FIG. 4) and an external tube (not illustrated) and forms a respiratory channel for a patient, an interior microphone 170 that is attached to an inner wall of the respiratory channel of the body 2 and extracts a respiratory sound of the patient, a flow rate sensor 180 that is attached to the inner wall of the respiratory channel of the body 2 and measures a respiratory flow rate of the patient, and a suction alarm control unit 300 that extracts a frequency of the respiratory sound of the interior microphone 170 based on information of the respiratory flow rate, analyzes frequency characteristics of the respiratory sound, and then outputs a phlegm suction or internal tube replacement alarm signal when the frequency and an abnormal respiratory cycle or flow rate data which are stored in advance are compared and are equal to each other within a certain frequency or amplitude range.

The body 2 can be configured to include a tracheostomy tube coupling flange 100 that has a cylindrical flange 120 which is coupled to the entrance 20 of the tracheostomy tube 10 and one or more support flanges 110 which project from an outer surface of the cylindrical flange 120 and fix and support the body 2 on a tracheostomy tube flange 30 of the tracheostomy tube10, and an external tube inserting pipe 200 which extends from the cylindrical flange 120, into which an external tube is inserted and coupled, and which forms the respiratory channel.

The body 2 can be configured to further include at least one of an LED unit 140 that emits light for a suction or internal tube replacement alarm; and a speaker unit 150 that outputs a sound for a suction or internal tube replacement alarm.

In addition, the body 2 can be configured to further include a power button 130 for power on and off and a charging terminal 190 for charging an internal battery.

The interior microphone 170 is mounted perpendicularly to air flow so as to minimize a frictional sound against air flowing in and out by respiration and enable full respiratory sound analysis to be conducted.

An environment measured by the interior microphone 170 include inhalation and exhalation environments, and humidity of about 95% is measured during the inhalation. Hence, the phlegm suction alarm issuing apparatus 1 can be configured to further include a waterproof member that seals the interior microphone 170 in order to waterproof the interior microphone 170.

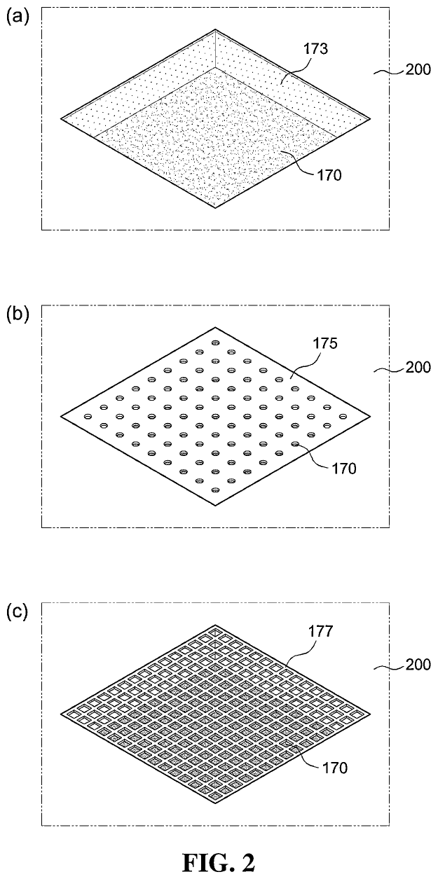

FIG. 2 is a view illustrating the waterproof member of the interior microphone 170 in FIG. 1.

As illustrated in FIG. 2, the waterproof member can be a waterproof membrane 173 which transmits vibration of the respiratory sound and blocks moisture, as illustrated in (a) of FIG. 2. As illustrated in (b) and (c) of FIG. 2, the waterproof member can be a waterproof hole plate 175 or a waterproof mesh plate 177 which has holes or grid holes having a diameter of about 1 μm or smaller through which no water droplet passes.

In addition, as illustrated in FIG. 1, the phlegm suction alarm issuing apparatus 1 can be configured to further include an exterior microphone 160 that is attached to an exterior part of the body 2 and extracts an exterior voice sound. Further, the suction alarm control unit 300 can be configured to remove noise contained in the respiratory sound of the patient by removing the exterior voice sound extracted by the exterior microphone from the respiratory sound extracted by the interior microphone.

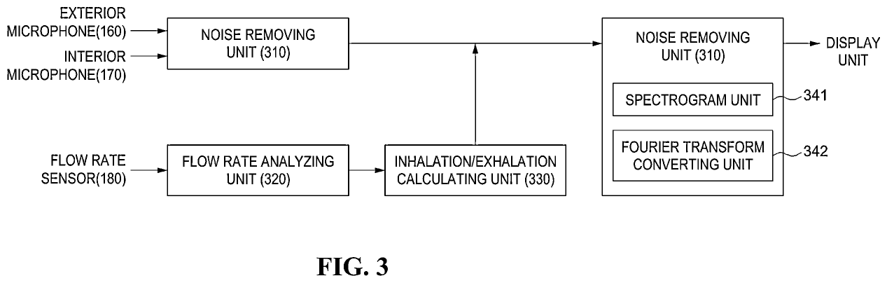

FIG. 3 is a functional block diagram of the suction alarm control unit 300 in FIG. 1.

As illustrated in FIG. 3, the suction alarm control unit 300 can be configured to include a noise removing unit 310, a flow rate analyzing unit 320, an inhalation/exhalation calculating unit 330, and a respiration signal analyzing unit 340.

The noise removing unit 310 in the configuration collects a sound signal of the exterior microphone 160 and a sound signal of the interior microphone 170 and then performs active noise cancelling by adding, to the sound signal of the interior microphone, a sound signal having an opposite phase of an amplitude equal to an amplitude of the sound signal of the exterior microphone 160. In this manner, the exterior noise contained in the sound signal detected by the interior microphone 170 is minimized.

The flow rate analyzing unit 320 is configured to be linked to the flow rate sensor 180 and detect a respiratory cycle, a respiratory flow rate, a flow direction of the patient.

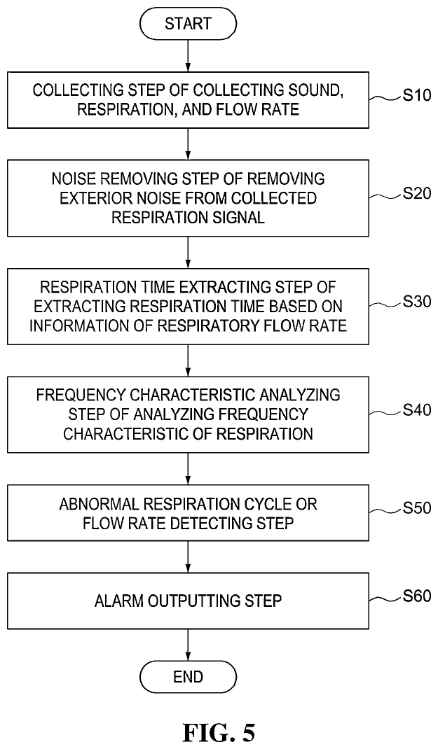

The inhalation/exhalation calculating unit 330 enables both accurate start time and finish time of inhalation and exhalation of the patient's respiration based on information of the respiratory cycle, the respiratory flow rate, and the flow direction acquired by the flow rate analyzing unit 320. Further, the inhalation/exhalation calculating unit is configured to perform data processing and analysis based on the inhalation time for all of the respiration cycles of the patient. With reference to FIG. 5 to be described below, in general, it is difficult to discriminate between inhalation and exhalation based on a magnitude of a simple sound signal when a patient has a low respiratory sound; however, flow rate data enables the inhalation and the exhalation to be easily discriminated.

The respiration signal analyzing unit 340 is configured to include a spectrogram unit 341 and a Fourier transform unit 343.

The respiration signal analyzing unit 340 is configured to output, as a graph on a display unit, a signal magnitude based on time, a spectrogram visually displaying a change in frequency based on time through the short-term Fourier transform (STFT), and a signal magnitude based on a frequency converted through the fast Fourier transform (FFT) according to each type of respiratory sound.

When the phlegm suction alarm issuing apparatus 1 does not include the exterior microphone, the noise removing unit 310 can be substituted only by an amplifying means that amplifies the respiratory sound of the interior microphone 170.

FIG. 4 is a view illustrating a procedure of coupling the phlegm suction alarm issuing apparatus 1 for a tracheostomy patient to the tracheostomy tube 10 according to the embodiment of the present invention. (a) of FIG. 4 illustrates a state before the phlegm suction alarm issuing apparatus 1 is coupled to the tracheostomy tube 10 according to the embodiment of the present invention. (b) of FIG. 4 illustrates a state after the phlegm suction alarm issuing apparatus 1 is coupled to the tracheostomy tube 10 according to the embodiment of the present invention.

The phlegm suction alarm issuing apparatus 1 according to the embodiment of the present invention having the above-described configuration can detect a change in respiratory sound due to phlegm in the trachea. In addition, according to the present invention, the microphone and the flow rate sensor are buried not to interfere with the respiratory channel, and thereby accurate respiration is measured with stability. Hence, accuracy of the phlegm suction alarm signal is improved.

In addition, the phlegm suction alarm issuing apparatus 1 of the present invention is designed to have a battery or an electronic device such as the suction alarm control unit 300 to be positioned at the support flange 110 which is near a human body, that is, the neck, at a side of the external entrance 20 of the tracheostomy tube 10 such that a minimum physical load is imposed on the patient. In addition, the phlegm suction alarm issuing apparatus 1 enables more accurate sound analysis to be conducted even in an environment in which external noise is very much produced by using the active noise cancelling, the waterproof member, and the flow rate sensor.

FIG. 5 is a flowchart illustrating a processing procedure of a phlegm suction alarm issuing method according to another embodiment of the present invention.

According to the other embodiment of the present invention, the phlegm suction alarm issuing method can be configured to include a collecting step S10, a respiration time extracting step S30, a frequency characteristic analyzing step S40, an abnormal respiration or flow rate detecting step S50, and an alarm outputting step S60 which are executed by the suction alarm control unit 300, the phlegm suction alarm issuing method being performed by the phlegm suction alarm issuing apparatus 1 including the body 2 having the respiratory channel for a patient, the interior microphone 170 and the flow rate sensor 180 attached to the respiratory channel, and the suction alarm control unit 300 which analyzes a respiratory sound extracted by the interior microphone 170 and a respiratory flow rate extracted by the flow rate sensor and outputs a phlegm suction or internal tube replacement alarm.

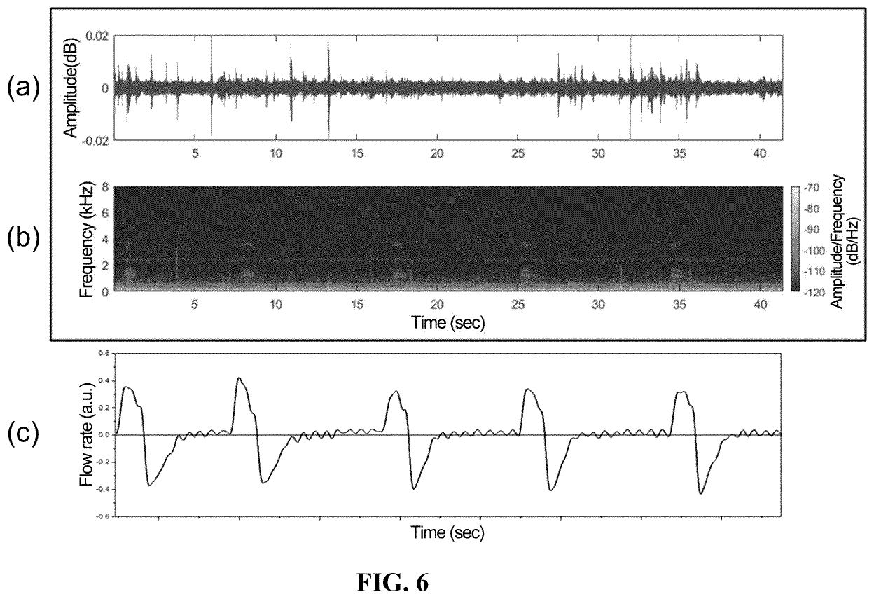

In the collecting step S10, a processing procedure of detecting and collecting the respiratory sound and the respiratory flow rate of the patient through the respiratory channel by the suction alarm control unit 300 using the interior microphone 170 and the flow rate sensor 180 is performed. FIG. 6 is a graph illustrating (a) sound signal, (b) spectrogram, and (c) flow rate signal which are collected in the collecting step S10 in FIG. 5. Through the execution of the collecting step S10, a graph showing (a) sound signal, (b) spectrogram, and (c) flow rate signal of the respiration of the patient can be obtained as illustrated in FIG. 6.

In the respiration time extracting step S30, a processing procedure of extracting a respiration time from the respiratory sound based on information of the respiratory flow rate by the suction alarm control unit is performed. In other words, when the respiratory flow rate information in (c) of FIG. 6 to be described below is synchronized with the respiratory sound, the inhalation and exhalation cycle of the respiration of the patient can be accurately extracted with the respiration time.

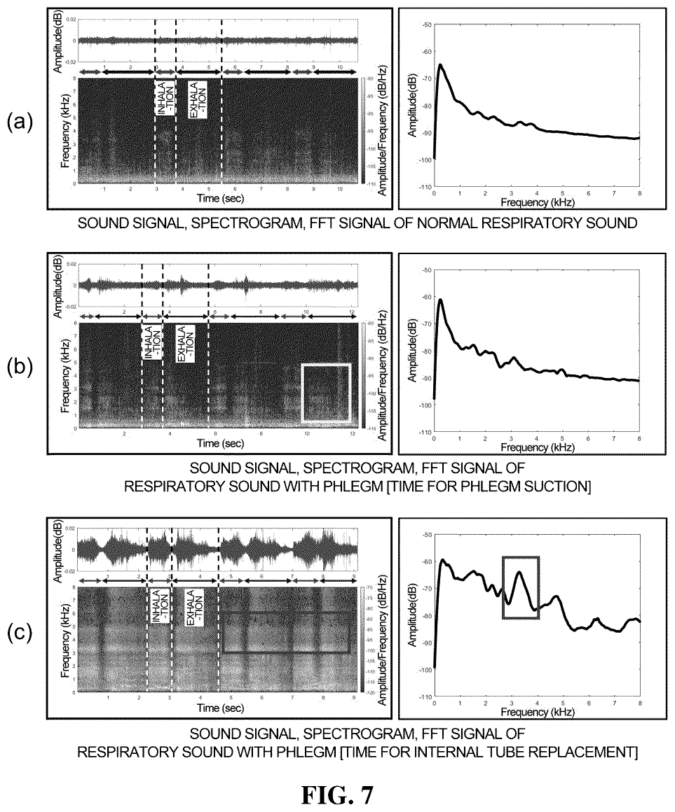

In the frequency characteristic analyzing step S40, a graph illustrating analysis of a signal magnitude based on time of respiration of the patient, a spectrogram visually displaying a change in frequency based on time through the short-term Fourier transform (STFT), and a signal magnitude based on a frequency converted through the fast Fourier transform (FFT) is displayed. FIG. 7 is a graph illustrating (a) signal magnitude based on time, (b) spectrogram visually displaying a change in frequency based on time through the short-term Fourier transform (STFT), and (c) signal magnitude based on a frequency converted through the fast Fourier transform (FFT) according to each type of respiratory sound.

In the abnormal respiration or flow rate detecting step S50, a processing procedure of detecting an abnormal respiration cycle or flow rate is performed, when analysis information of the frequency characteristic of the respiration analyzed in the frequency characteristic analyzing step and analysis information of a frequency characteristic corresponding to phlegm suction or internal tube replacement stored in advance are compared and are equal to each other within a certain frequency or amplitude range.

In other words, the normal respiration in (a) of FIG. 7 is characterized by having a sound signal which is not clearly discriminated from noise. On the other hand, respiration for which phlegm suction needs to be performed in (b) of FIG. 7 has a relatively larger signal magnitude compared to the normal respiration. Further, repeated vertical lines are displayed on the spectrogram due to trembling of phlegm attached to the trachea (rectangular region in (b) of FIG. 7). As illustrated in (c) of FIG. 7, the respiration for which internal tube replacement needs to be performed has an increase in overall signal magnitude and has a large signal in a high-frequency region (rectangular region in (c) of FIG. 7).

In the alarm outputting step S60, an alarm of the suction time or internal tube replacement time is output when an abnormal respiration or flow rate based on (b) or (c) of FIG. 7 is detected in the abnormal respiration or flow rate detecting step S50. In this case, the alarm can be output as the LED light emission, a buzzer sound, or the like.

In addition, although not illustrated, the phlegm suction alarm issuing apparatus 1 of the present invention can be configured to have a near field communication means or a mobile communication means so as to output a phlegm suction alarm signal to a terminal such as a mobile phone of a medical team or a guardian at a remote location. When the terminal is a mobile phone, an additional suction alarm application can be installed. Further, when the terminal is a medical apparatus having an internal communication means, the terminal can be configured to output the phlegm suction alarm signal transmitted from the phlegm suction alarm issuing apparatus 1 as the LED, a buzzer sound, or the like. In this case, the phlegm suction alarm issuing apparatus 1 can be configured to have individual identifiers to be identified by respective medical teams or guardians at remote locations.

The phlegm suction alarm issuing apparatus 1 may be configured to further include the exterior microphone that is attached to the exterior part of the body and extracts the exterior voice sound. In this case, the phlegm suction alarm issuing method can be configured to further include, after the collecting step S10, the noise removing step S20 of removing noise contained in the respiratory sound of the patient by the suction alarm control unit 300, by removing the exterior voice sound extracted by the exterior microphone 160 from the respiratory sound extracted by the interior microphone 170.

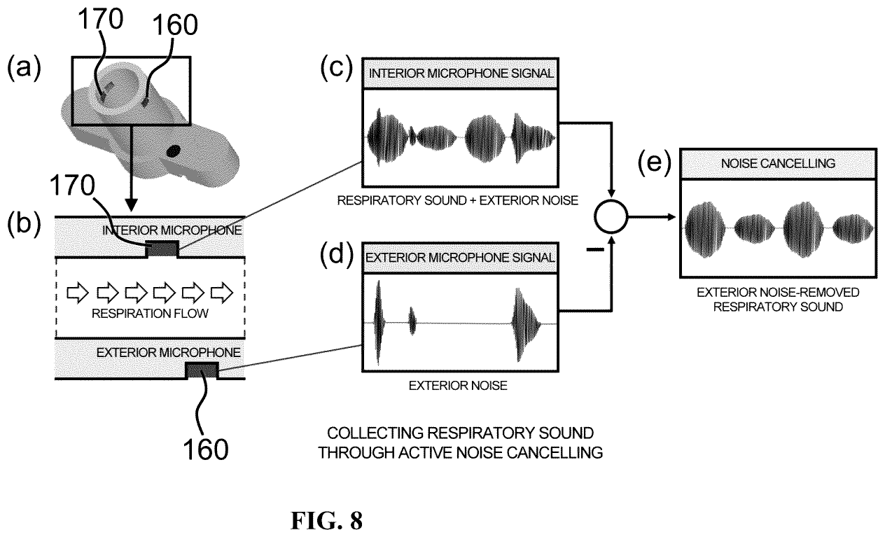

FIG. 8 is a view illustrating the noise removing step S20 performed by the noise removing unit 310 in FIG. 5.

In the noise removing step S20, a sound signal of the exterior microphone 160 and a sound signal of the interior microphone 170 are collected through the exterior microphone 160 and the interior microphone 170 of the phlegm suction alarm apparatus 1, as illustrated in (a) and (b) of FIG. 8. Then, the active noise cancelling ((e) of FIG. 8) is performed by adding a sound signal ((d) of FIG. 8) having an opposite phase of an amplitude equal to an amplitude of the sound signal of the exterior microphone 160 to the sound signal ((c) of FIG. 8) of the interior microphone.

The technical idea of the present invention described above is described specifically in the preferred embodiment; however, note that the embodiment is provided for the description and is not provided to limit the present invention thereto. In addition, it is possible for a person of ordinary knowledge in the technical field of the present invention to make various embodiments within the scope of the technical idea of the present invention. Consequently, an actual scope of technical protection of the present invention is to be determined based on technical ideas of the accompanying claims.

1. A phlegm suction alarm apparatus comprising:
a body that is coupled to an entrance of a tracheostomy tube and an external tube and forms a respiratory channel for a patient;
an interior microphone that is attached to an inner wall of the respiratory channel of the body and configured to extract a respiratory sound of the patient;
a flow rate sensor that is attached to the inner wall of the respiratory channel of the body and configured to measure a respiratory flow rate of the patient; and
a suction alarm control unit that is configured to extract a frequency of the respiratory sound of the interior microphone based on information of the respiratory flow rate, analyze frequency characteristics of the respiratory sound, and then output a phlegm suction or internal tube replacement alarm signal when the frequency and an abnormal respiratory cycle or flow rate data which are stored in advance are compared and are equal to each other within a certain frequency or amplitude range.

2. The phlegm suction alarm issuing apparatus according to claim 1, further comprising:
a waterproof member that seals the interior microphone in order to waterproof the interior microphone.

3. The phlegm suction alarm issuing apparatus according to claim 1, further comprising:
an exterior microphone that is attached to an exterior part of the body and configured to extract an exterior voice sound,
wherein the suction alarm control unit is configured to remove noise contained in the respiratory sound of the patient by removing the exterior voice sound extracted by the exterior microphone from the respiratory sound extracted by the interior microphone.

4. The phlegm suction alarm issuing apparatus according to claim 1,
wherein the body includes:
a tracheostomy tube coupling flange that has a cylindrical flange which is coupled to the entrance of the tracheostomy tube and one or more support flanges which project from an outer surface of the cylindrical flange and support the body on the tracheostomy tube; and
an external tube inserting pipe which extends from the cylindrical flange and into which an external tube is inserted and coupled.

5. The phlegm suction alarm issuing apparatus according to claim 1,
wherein the body includes at least one of:
an LED unit that emits light for a suction or internal tube replacement alarm; and
a speaker unit that outputs a sound for a suction or internal tube replacement alarm.

6. The phlegm suction alarm issuing apparatus according to claim 1,
wherein the suction alarm control unit includes:
a noise removing unit that collects a sound signal of the exterior microphone and a sound signal of the interior microphone and then performs active noise cancelling by adding, to the sound signal of the interior microphone, a sound signal having an opposite phase of an amplitude equal to an amplitude of the sound signal of the exterior microphone;
a flow rate analyzing unit that is linked to the flow rate sensor and detects a respiratory cycle, a respiratory flow rate, a flow direction of the patient;
an inhalation/exhalation calculating unit that enables both accurate start time and finish time of inhalation and exhalation of the patient's respiration based on information of the respiratory cycle, the respiratory flow rate, and the flow direction of the flow rate analyzing unit; and
a respiration signal analyzing unit that outputs, as a graph on a display unit, a signal magnitude based on time, a spectrogram visually displaying a change in frequency based on time through the short-term Fourier transform (STFT), and a signal magnitude based on a frequency converted through the fast Fourier transform (FFT) according to each type of respiratory sound.

7. A phlegm suction alarm issuing method that is performed by a phlegm suction alarm issuing apparatus including a body having a respiratory channel for a patient, an interior microphone and a flow rate sensor attached to the respiratory channel, and a suction alarm control unit which analyzes a respiratory sound extracted by the interior microphone and a respiratory flow rate extracted by the flow rate sensor and outputs a phlegm suction or internal tube replacement alarm, the phlegm suction alarm issuing method comprising:
a collecting step of detecting and collecting the respiratory sound and the respiratory flow rate of the patient through the respiratory channel by the suction alarm control unit using the interior microphone and the flow rate sensor;
a respiration time extracting step of extracting a respiration time from the respiratory sound based on information of the respiratory flow rate by the suction alarm control unit;
a frequency characteristic analyzing step of analyzing a frequency characteristic of respiration;
an abnormal respiration or flow rate detecting step of detecting an abnormal respiration cycle or flow rate when analysis information of the frequency characteristic of the respiration analyzed in the frequency characteristic analyzing step and analysis information of a frequency characteristic corresponding to phlegm suction or internal tube replacement stored in advance are compared and are equal to each other within a certain frequency or amplitude range; and
an alarm outputting step of outputting an alarm of the suction time or internal tube replacement time, when an abnormal respiration or flow rate is detected in the abnormal respiration or flow rate detecting step.

8. The phlegm suction alarm issuing method according to claim 7,
wherein the phlegm suction alarm issuing apparatus further includes: an exterior microphone that is attached to an exterior part of the body and extracts an exterior voice sound, and
wherein the phlegm suction alarm issuing method further comprises: after the collecting step, a noise removing step of removing noise contained in the respiratory sound of the patient by the suction alarm control unit by removing the exterior voice sound extracted by the exterior microphone from the respiratory sound extracted by the interior microphone.
